(19) United States
(12) Patent Application Publication
(10) Pub. No.: US 2020/0086066 A1
(43) Pub. Date: Mar. 19, 2020

(54) DEVICES AND METHODS FOR SENSOR-ENHANCED NEEDLE PLACEMENT

(71) Applicants: Brendan Riordan, Rockport, ME (US); Alexander Abess, Rockport, ME (US); Travis Emery, Portland, ME (US); Annie Hang, Belmont, MA (US); Katelyn Jones, Atlanta, GA (US); Kelly Anne McElreath, Oakland, MI (US); Jack Kirsch, Concord, NH (US); Rockport Medical Technologies LLC, Rockport, ME (US)

(72) Inventors: Brendan RIORDAN, Rockport, ME (US); Alexander ABESS, Rockport, ME (US); David MORRILL, Gorham, ME (US); Travis EMERY, Portland, ME (US); Annie HANG, Belmont, MA (US); Katelyn JONES, Atlanta, GA (US); Kelly Anne MCELREATH, Oakland, MI (US); Jack KIRSCH, Concord, NH (US)

(21) Appl. No.: 16/610,357

(22) PCT Filed: May 3, 2018
(86) PCT No.: PCT/US2018/030918

§ 371 Date: Nov. 1, 2019

Related U.S. Application Data
(60) Provisional application No. 62/500,656, filed on May 3, 2017.

(51) Int. Cl. A61M 5/48; A61M 25/06; A61M 1/00; A61M 19/00

(52) U.S. Cl. CPC A61M 5/486; A61M 25/0606; A61M 1/008; A61M 5/365; A61M 25/0693; A61M 19/00; A61M 2205/3313; A61M 25/0618

(57) ABSTRACT

The invention features devices that measure the pressure of an injection or intrinsic physiologic pressures generated and measures the fluid content, e.g., blood content, of aspirated fluid during medical interventions requiring hypodermic needles, e.g., fluid sampling or medical injection procedures. The devices feature pressure and fluid content, e.g., blood, sensors that trigger indicators to alert the user of an issue during the procedure(s). The invention features a flexible cover for devices of the invention to facilitate introduction of a device into a sterile field. The invention also provides methods of using such devices and covers.

### FIELD OF THE INVENTION

The invention is directed to the field of fluid injection during medical and other procedures.

### BACKGROUND OF THE INVENTION

The introduction of a therapeutic fluid in close proximity to sensitive biological tissues in a human or animal, such as nerves of the peripheral nervous system, is a common medical practice. For example, Peripheral Nerve Block (PNB) is a medical procedure involving the introduction of a fluid in close proximity to nerves of the peripheral nervous system. The procedure is intended and commonly deployed in order to interrupt and/or reduce the magnitude of the pain response before, during, and/or after numerous surgical interventions as well as for pain management purposes outside of the surgical realm. Real-time ultrasonography is commonly and increasingly used to help visualize the tissue to be treated as well as needle tip position in relation to nearby tissues of interest including nerve bundles, lymph nodes, muscles, tendons, fascia, vascular structures, and other biological tissues. Various technologies and needle manufacturing techniques are employed with the goal of improving visibility of the needle with point-of-care ultrasonography to help ensure proper needle tip position and therapeutic fluid placement relative to these tissues of interest. Clinicians administering injections into biological tissues usually endeavor to confirm needle tip position relative to other nearby structures prior to injection in order to maximize procedure efficacy while avoiding adverse clinical outcomes, such as toxicity of the therapeutic fluid and damaging of sensitive tissues.

One such adverse outcome is Local Anesthetic Systemic Toxicity (LAST). LAST is generally associated with unintended intravascular access and introduction of anesthetic into the blood stream, and can result in symptoms and complications ranging from agitation, tachycardia, and delayed/aborted surgical procedures, to seizure, cardiopulmonary collapse, and death. Although LAST events are relatively infrequent, best practice tools, techniques, and training for avoiding and managing LAST events are a key component of standard of care regional anesthesia administration. Anesthesiologists typically practice techniques for LAST avoidance including pre-injection aspiration and inspection for blood, as well as confirmation of visible flow of the injectant on the ultrasound monitor if ultrasound is being used. The effectiveness of these subjective patient safety techniques varies according to access to the requisite equipment and advanced sonography (“sonoanatomy”) training and skill. Human errors are common and contribute to imperfect safety monitoring. Taken together, despite the best efforts of clinicians and the assistance of ultrasound, LAST events still occur with a reported frequency in the range of 1 in 5,000 to 1 in 10,000. Many believe that the true rate of LAST is much higher, as reporting is voluntary. Considering the large number of local anesthesia administration procedures performed worldwide every day, this statistic is highly significant.

The second adverse outcome due to improper needle tip placement is commonly referred to as nerve injury. Nerve injury is currently understood to result from multiple potential causes, one of which includes intraneural needle placement. The mechanism for nerve injury can be from direct laceration of nerve fibers via the needle, neural ischemia from excess pressure created by the injected local anesthetic, or direct chemical toxicity from the anesthetic itself. Current practice for avoiding nerve injury involves avoiding direct nerve penetration and monitoring syringe pressure by tactile sensation during injection. Higher pressures required for injection of local anesthetic may indicate intraneural (or sub-epineural) needle tip location. This technique of subjective pressure monitoring is commonly used in combination with point-of-care ultrasound imaging. Neural tissue is known to be less compliant than other soft tissues resulting in palpably excessive syringe plunger pressure while injecting liquid anesthetic during the nerve block. In practice this method is largely ineffective, highly subjective, and requires a highly trained clinician. It is further complicated by the fact that the sensation of the syringe plunger pressure varies significantly according to injection rate, syringe volume and diameter, needle gauge and length, as well as length and elasticity of the anesthetic conduction tubing.

Unintended injection into a biological structure can result from the inability to confirm the exact location of the tip of the needle. Even with ultrasound use, signal attenuation from soft tissue (increasing with target depth) and needle angle in combination with narrow ultrasound beam widths (on the order of 1 mm) make precise location of needle tip challenging, even in experienced hands.

Due to the increasing emergence of new medical technology and hand-held electronic devices, there is a growing need for a method to create sterile barriers between non-sterilizable components and a sterile procedure environment. Invasive surgical techniques, such as injection procedures, demand strict adherence to sterile guidelines to minimize the risk of infection for the patient. The common standard for sterility is the Sterility Assurance Level (SAL), which quantifies the probability of any microorganism surviving after sterilization. Sterilization regulations depend on the specific procedure being performed. Invasive medical devices are most commonly held to SAL 10⁻⁶, i.e., one non-sterile device per one million sterilized. Depending upon use of the device, SAL assurance of a lesser value is occasionally acceptable.

This sterilization standard is achieved with state-of-the-art sterilization techniques, which involve a variety of chemical and heat treatments depending on the specifications of the device. Heat based sterilization, either dry or wet, e.g., an autoclave, is used to clean many tools that can withstand high temperatures. Chemical methods, such as ethylene oxide gas and hydrogen peroxide gas, are used for devices that cannot withstand high temperatures, high pressures, or exposure to moisture. However, chemical gas sterilization methods can damage circuit boards, batteries, and other electronic elements, and thus cannot be performed on many battery powered medical devices. Designing an electronic device that can withstand sterilization presents many challenges, such as an increase in manufacturing and material costs so that the device can endure multiple sterilization cycles. In addition, devices must be manufactured water-tight to prevent the electronic components from being damaged. Even when medical devices with electronic components can be sterilized, the turnover time for sterilization necessitates the ownership of multiple devices if the device is needed for multiple procedures per day. Medical devices are often very expensive, presenting a budgetary restriction from buying multiple devices.

Existing low-cost disposable sterile covers allow expensive electronic components to be reused without undergoing sterilization cycles. Medical device covers of the prior art that enter the sterile field primarily include covers made of a polymeric material, often non-elastic polyurethane, with one sealed end, which enters the field and interacts with the patient, and one open end, into which the device is slipped into and tied off at, or kept at a sufficient distance from, the sterile field. For example, encapsulating an ultrasound probe with a thin plastic sleeve allows clinicians to bring the non-sterile probe into a sterile field. The sterile plastic sleeve is long enough to cover the probe until it exits the sterile field. Such a cover suffices for corded devices which do not require pass through of solutions or device components into the subject; however, these covers are limited in function by their closed-end design.

There exists a high level of clinical awareness of these significant challenges within the medical field, with respect to both injection monitoring and sterile use of non-sterilizable medical devices. Despite the known challenges, no technology or device yet exists to objectively and reliably automate opening injection pressure monitoring, confirmation of aspiration, and aspirated blood detection, e.g., while also providing for a sterile injection environment to minimize potential contamination. There is, therefore, a need for methods and devices that codify clinician expertise and combine these functions in such a manner as to allow rapid identification of those events associated with the complications of toxicity and unintended injury to nearby structures, e.g., while maintaining a sterile patient environment.

### SUMMARY OF THE INVENTION

We have developed a device-based monitoring system that provides real-time information about the precise and accurate placement of needles during diagnostic interrogation and therapeutic intervention procedures. We have further developed a sterile cover for a medical device, e.g., the device-based monitoring system of the invention, that provides a cover for a non-sterile portion of the device such that it can safely be used during diagnostic interrogation and therapeutic intervention procedures.

In one embodiment, such a procedure can involve a PNB, where an anesthetic is injected in close proximity to a nerve bundle to reduce pain in a specific region of the body. In other embodiments, the procedure is a nerve block where the injection site is not adjacent to a nerve, e.g., injection into a tissue plane or compartment through which the nerve passes. In other embodiments, the pressure measured is a waveform, such as a pulsatile pressure waveform or vascular pressure waveform. The invention employs a pressure and/or fluid content, e.g., blood, sensor for measuring the characteristics of an injection or aspiration, enabling the clinician to make adjustments as needed based on the results of an indicator.

In one embodiment, the invention relates to the administration of anesthetic during PNB procedures and efforts to increase needle placement accuracy and thereby achieve patient safety and procedure efficacy improvements. The invention has direct applicability to additional diagnostic and therapeutic clinical interventions such as those involving transcutaneous, intravascular (venous and arterial), intrathecal, epidural and other intracompartmental introduction and sampling of fluids, and the delivery of catheters and other surgical instruments. These additional applications include but are not limited to various types of epidural catheters and injections, lumbar punctures, and arterial or venous catherization, e.g., central venous catheter placement.

In one aspect, the invention features a device for measuring fluid properties containing a housing having a first lumen with a proximal opening and a distal opening, a hypodermic needle having a second lumen permanently affixed to the housing so that the first and second lumens are fluidically connected via the distal opening, and a pressure sensor operatively coupled to the first or second lumen to measure fluid pressure in the first or second lumen. In some embodiments, the hypodermic needle includes a catheter releasably connected to the needle. In some embodiments, the device further includes a fluid conducting source which is fluidically connected to the proximal opening of the first lumen. In further embodiments, the device contains circuitry configured to process data collected by the pressure sensor and provide an indicator signal. In other embodiments, the device contains circuitry configured to collect, store, and/or transmit data collected by the pressure sensor. In some embodiments, the pressure sensor is an inductive, resistive, piezoelectric, or capacitive transducer, e.g., a pressure sensor.

In some embodiments, the device further contains a fluid content, e.g., blood, sensor operatively coupled to the lumen, e.g., to determine the presence or amount of blood in the lumen. In some embodiments, the fluid content, e.g., blood, sensor is an optical sensor, e.g., which contains a light detector and light source. In other embodiments, the fluid content, e.g., blood, sensor is an electrical sensor.

In any of the above embodiments, the device may include an indicator actuated by the indicator signal. In some embodiments, the indicator is an audible, visual, or tactile indicator. In other embodiments, the indicator signal is transmitted externally.

In another aspect, the invention features a device for monitoring fluid properties containing a housing having a lumen with a proximal opening and distal opening and a fluid content, e.g., blood, sensor, operatively coupled to the lumen, e.g., to determine the presence or amount of blood in the lumen. In some embodiments, the device contains a hypodermic needle having a second lumen so that the first and second lumens are fluidically connected via the distal opening. The hypodermic needle may be permanently affixed to the housing. Alternatively, the hypodermic needle is releasably affixed to the housing. In some embodiments, the hypodermic needle includes a catheter releasably connected to the needle. In some embodiments, the fluid content, e.g., blood, sensor is an optical sensor, e.g., which contains a light detector and light source. In other embodiments, the fluid content, e.g., blood, sensor is an electrical sensor. In further embodiments, the device contains circuitry configured to process data collected by the fluid content, e.g., blood, sensor and provide an indicator signal. In further embodiments, the device contains circuitry configured to collect, store, and/or transmit data collected by the fluid content, e.g., blood, sensor. In some embodiments, the device contains a pressure sensor operatively coupled to the lumen to measure fluid pressure in the lumen. In some embodiments, the pressure sensor is an inductive, resistive, piezoelectric, or capacitive transducer, e.g., a piezoresistive transducer. In further embodiments, the device contains circuitry configured to process data collected by the pressure sensor and provide an indicator signal. In further embodiments, the device contains a fluid content, e.g., blood, and/or pressure indicator actuated by the fluid content, e.g., blood, and/or pressure indicator signal. In other embodiments, the fluid content, e.g., blood, and/or pressure indicator signal is transmitted externally. In any of the above embodiments, the device includes a fluid conducting source which is fluidically connected to the proximal opening of the first lumen.

In another aspect, the invention features a kit containing an inner housing having a first lumen with a proximal opening and distal opening and a pressure sensor operatively connected to the first lumen to measure fluid pressure in the first lumen and/or a fluid content, e.g., blood, sensor operatively connected to the lumen, e.g., to measure the presence or amount of blood in the first lumen, and an outer housing containing circuitry electrically configured to process data collected by the pressure or fluid content, e.g., blood, sensor and provide an indicator signal. The inner housing mates with the outer housing to provide connectivity between the pressure or fluid content, e.g., blood, sensor and the circuitry. In some embodiments, the inner housing contains a hypodermic needle having a second lumen so that the first and second lumens are fluidically connected via the distal opening. In further embodiments, the kit includes a hypodermic needle having a second lumen and being connectable to the first lumen via the distal opening. In certain embodiments, the inner housing contains both the pressure sensor and the fluid content, e.g., blood, sensor. The fluid content, e.g., blood, sensor may be an optical sensor, e.g., having a light detector and a light source. In other embodiments, the fluid content, e.g., blood, sensor includes an electrical sensor. In further embodiments, the inner or outer housing contains an indicator actuated by the indicator signal. The indicator is, for example, a visual, tactile, and/or audible indicator. In some embodiments, the circuitry transmits the indicator signal externally. In other embodiments, the circuitry is configured to collect, store, and/or transmit data collected by the pressure or fluid content, e.g., blood, sensor. The pressure sensor may be an inductive, resistive, piezoelectric, or capacitive transducer, e.g., a piezoelectric transducer. In further embodiments, the kit includes a fluid conducting source that is fluidically connectable to the proximal opening of the first lumen.

In a related aspect, the invention features a method for measuring pressure during a medical procedure, e.g., a PNB, using a device or kit of the invention. In one embodiment, the method includes providing a device having a housing having a first lumen with a proximal opening and a distal opening; a hypodermic needle having a second lumen attached to the housing so that the first and second lumens are fluidically connected via the distal opening; a pressure sensor operatively coupled to the first or second lumen to measure fluid pressure in the first or second lumen; and circuitry configured to process data collected by the pressure sensor and provide an indicator signal thereof; inserting the hypodermic needle into a site of the medical procedure; and measuring the pressure in the first or second lumen during the medical procedure via the pressure sensor to produce data wherein the circuitry analyzes the data and generates the indicator signal representative of the pressure.

In some embodiments, the method produces an indicator signal when the pressure is at or above a threshold pressure. In other embodiments, the pressure is the opening injection pressure. In other embodiments, the pressure is from a pressure waveform, such as a pulsatile pressure waveform or vascular pressure waveform.

In another related aspect, the invention features a method for the detection of fluid content, e.g., blood, during a medical procedure, e.g., PNB, using a device or kit of the invention. In one embodiment, the method includes providing a device having a housing having a first lumen with a proximal opening and a distal opening; a hypodermic needle having a second lumen attached to the housing so that the first and second lumens are fluidically connected via the distal opening; a fluid content, e.g., blood, sensor operatively coupled to the first or second lumen, e.g., to measure the presence or amount of blood in the first or second lumen; and circuitry configured to process data collected by the fluid content, e.g., blood, sensor and provide an indicator signal thereof; inserting the hypodermic needle into a site of the medical procedure; aspirating fluid from the site via the hypodermic needle; and measuring fluid content, e.g., for the presence or amount of blood in the aspirated fluid via the fluid content, e.g., blood, sensor to produce data wherein the circuitry analyzes the data and generates the indicator signal representative of the fluid content, e.g., presence or amount of blood.

In yet another related aspect, the invention features an apparatus comprising a constrained flexible cover for a structure; an application assistance structure connected to the constrained flexible cover, where removal of the application assistance structure allows the constrained flexible cover to assume substantially the same shape as the structure. In some embodiments, the constrained flexible cover is sterile. In other embodiments, the application assistance structure is sterile. In some embodiments, the constrained flexible cover includes a polymer, e.g., selected is selected from the group consisting of silicone, polyurethane, polytetrafluoroethylene, expanded PTFE, fluorinated ethylene propylene, perfluoroalkoxy, ethylene tetrafluoroethylene, polyvinylidene fluoride, polyethylene, and polypropylene, in particular silicone. In certain embodiments, the application assistance structure includes an opening sized to permit the structure to pass. In some embodiments, the application assistance structure is releasably connected to the flexible cover. In some embodiments, the structure is a medical device, in particular a medical device disclosed herein.

In yet another related aspect, the invention features a method of covering a structure, by providing an apparatus that includes a constrained flexible cover for the structure and an application assistance structure connected or connectable to the constrained flexible cover; providing the structure; inserting one end of the structure into the application assistance structure; and moving the application assistance structure to deploy the constrained flexible cover to assume substantially the same shape as the structure. In some embodiments, the constrained flexible cover is sterile. In other embodiments, the application assistance structure is sterile. In some embodiments, the constrained flexible cover includes a polymer, e.g., selected from the group consisting of silicone, polyurethane, polytetrafluoroethylene, expanded PTFE, fluorinated ethylene propylene, perfluoroalkoxy, ethylene tetrafluoroethylene, polyvinylidene fluoride, polyethylene, and polypropylene, in particular silicone. In certain embodiments, the application assistance structure includes an opening sized to permit the structure to pass. In some embodiments, the application assistance structure is releasably connected to the flexible cover. In some embodiments, the structure is a medical device, in particular a medical device disclosed herein.

In yet another related aspect, the invention features a kit including a structure; a constrained flexible cover for the structure; and an application assistance structure connected or connectable to the constrained flexible cover, where removal of the application assistance structure allows the constrained flexible cover to assume substantially the same shape as the structure. In some embodiments, the structure is a medical device, in particular a medical device disclosed herein. In some embodiments, the constrained flexible cover is sterile. In other embodiments, the application assistance structure is sterile. In some embodiments, the constrained flexible cover includes a polymer, e.g., selected from the group consisting of silicone, polyurethane, polytetrafluoroethylene, expanded PTFE, fluorinated ethylene propylene, perfluoroalkoxy, ethylene tetrafluoroethylene, polyvinylidene fluoride, polyethylene, and polypropylene, in particular silicone. In certain embodiments, the application assistance structure includes an opening sized to permit the structure to pass.

### BRIEF DESCRIPTION OF THE DRAWINGS

FIGS. 1A-1D: Isometric views of an embodiment of a device according to the invention showing the inner housing geometry and configuration of the light tunnel, pressure port, needle shaft, alignment groove, and attachment elements. FIGS. 1A and 1B are overall views of an embodiment of the device, and FIGS. 1C and 1D are horizontal and vertical cross-sections.

FIGS. 2A-2B: Isometric views of an embodiment of a device according to the invention showing the inner housing geometry and configuration of the light tunnel, pressure port, needle shaft, alignment groove, and attachment elements. FIG. 2A is a horizontal cross-section and FIG. 2B is a vertical cross-section which show an externally molded inner housing stop behind the hypodermic needle.

FIG. 3: Isometric view of an embodiment of a device according to the invention showing the inner housing geometry where the connection for a hypodermic needle has a flat backing structure and a securing element for a cover.

FIG. 4: Isometric view of an embodiment of a device according to the invention showing the inner housing geometry, attachment element, and fluid connections where a hypodermic needle is installed.

FIGS. 5A-5D: Isometric views of an embodiment of a device according to the invention showing an outer housing. FIGS. 5A and 5B are overall views of an embodiment of the device, and FIGS. 5C and 5D are horizontal and vertical cross-sections.

FIGS. 6A-6F: Isometric views of an embodiment of a device according to the invention showing inner and outer housings in the fully engaged position. FIGS. 6A and 6B are overall views of an embodiment of the device, and FIGS. 6C-6D and 6E-6F are horizontal and vertical cross-sections.

FIGS. 7A-7D: Isometric views of an embodiment of a device according to the invention showing an inner housing fully inserted into an outer housing. FIG. 7A is a vertical cross-section of the complete device, with FIG. 7B showing a zoomed in detail of the interface between the inner and outer housings. FIG. 7C is a horizontal cross-section of the complete device, with FIG. 7D showing a zoomed in detail of the fluid pathway and optical detection scheme.

FIGS. 8A-8D: Isometric views of an embodiment of a device according to the invention showing an outside surface of an outer housing with an inner housing inserted. FIG. 8A shows an outer housing with a cap containing a control button and status indicators. FIG. 8B is a zoomed in detail of indicator lights. FIG. 8C is a view of an inner housing inserted into an outer housing showing electrical connectors, with FIG. 8D showing a zoomed in detail of the electrical connectors.

FIG. 9: Isometric view of an embodiment of a complete device according to the invention with a rectangular outer housing with indicator lights. The dimensions of the outer housing and the installed hypodermic needle are shown.

FIG. 10: Block diagram of an embodiment of a complete device according to the invention, including electrical power, data transmission, user interface, and medical device.

FIGS. 11A-11B: Block diagrams of: A) a complete device according to the invention showing sterile and non-sterile portions of the device, and B) a complete device according to the invention within a cover. In FIG. 11B, 11.1 is the cover, 11.2 and 11.3 are points of access, and 11.4 and 11.5 are device components.

FIGS. 12A-12B: An application assistance structure installed over the distal end of a device according to the invention. The application assistance structure in FIG. 12A is ring shaped, and the side profile of the ring is shown in FIG. 12B.

FIG. 13: Photographs of an embodiment of a complete kit according to the invention, including an outer housing, and inner housing with a hypodermic needle, a flexible cover, and a ring-shaped application assistance structure.

FIGS. 14A-14B: An embodiment of a complete device according to the invention showing the relative size of an embodiment of a device according to the invention in the hand of a clinician.

FIG. 15: Photographs of an embodiment of a method according to the invention for applying a constrained flexible cover using an application assistance structure to a device according to the invention.

FIG. 16: Example output data from an embodiment of a device according to the invention showing the response of the optical system as a function of solution composition.

### DETAILED DESCRIPTION OF THE INVENTION

The invention provides devices for measuring pressure and/or determining fluid content, e.g., the presence or amount of blood, during a medical procedure, e.g., an injection. As one application, the devices are advantageous in providing real time feedback on the proper placement of a needle in a patient. Methods, including methods of using the devices, are also provided by the invention.

The invention provides a new method and device that integrates the typical elements of a needle configured for hypodermic introduction and sampling of fluids with circuitry, e.g., miniature and subminiature printed circuit board components including microelectromechanical sensors, microcontrollers, microprocessors, binary logic integrated circuits, power supplies, regulators, capacitors, charge controllers, amplifiers, transceivers, digital to analog converters, conductivity connectors, wireless connectivity modules, and others. The described system is generally intended to increase the amount of real time, point of care information available to clinicians and improve their ability to place needles with precision and accuracy relative to adjacent tissues of interest. The device function can include one or more of confirmation of aspiration, aspirated blood detection, opening injection pressure monitoring, physiologic pressure monitoring, communication of relevant measurements and alarm conditions to clinicians at the point of care in real time, and data collection and storage for administrative and clinical purposes as well as device refinement. The invention may also capture, store and transmit relevant data for post hoc analysis and evaluation, which can have important economic, legal and other consequences including clinician performance assessment, outcome-based reimbursements, and determination of liability in case of adverse events.

The invention further provides a flexible cover for a medical device, e.g., of the invention. This cover can be sterile and reduces the potential for contamination to the subject during a medical procedure by exposure to a portion of a medical device that is not sterilized. Methods, including methods for using the cover, are also provided by the invention.

### Devices

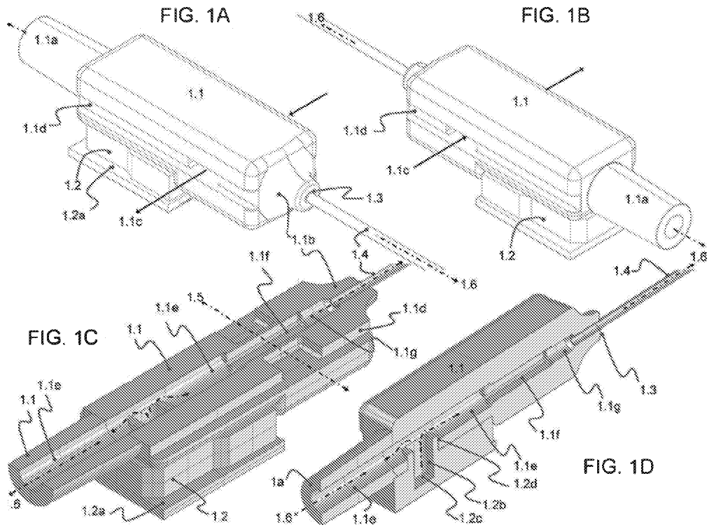

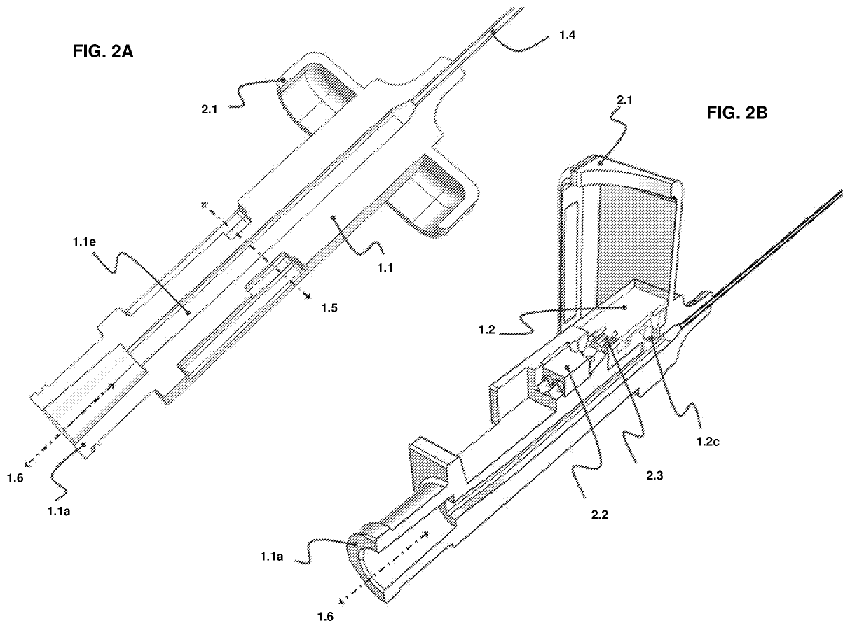

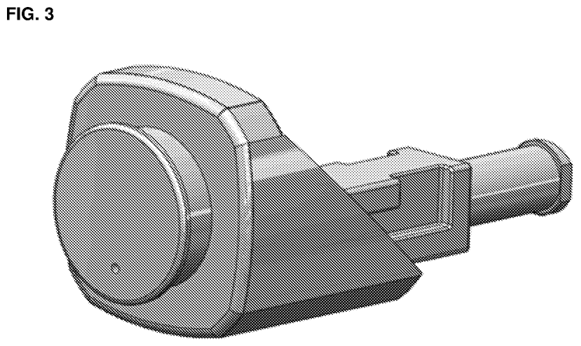

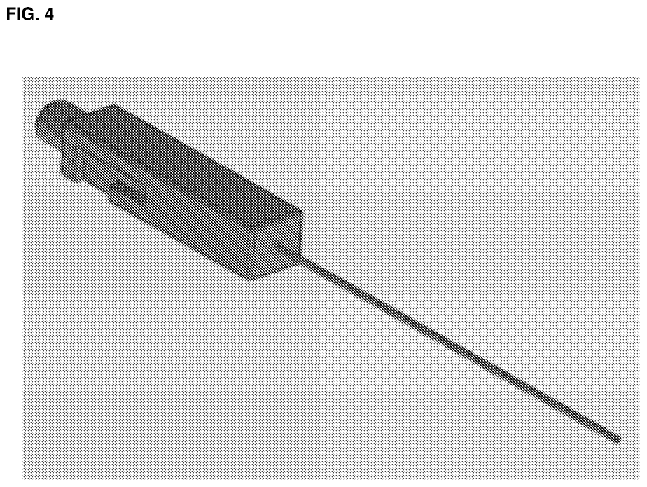

A device of the invention includes a housing including a lumen having a proximal opening and a distal opening. The housing of the lumen is configured to have a hypodermic needle permanently or releasably affixed to it such that the lumen of the needle and the lumen of the housing are connected, allowing fluid flow through the connected lumens. The proximal opening of the lumen is designed to be connected to a controllable fluid conducting source, such as a syringe barrel, tube, or other means of delivering a volume of fluid. The lumen of the housing is operatively connected to a sensor within the housing. The sensor is designed to collect data, e.g., pressure or fluid content, e.g., presence of blood, about the fluid as it is either injected into a specific biological tissue or aspirated from it. FIGS. 1A-1D show outer (FIGS. 1A and 1 ) and cross-section (FIGS. 1C and 1D) views of one embodiment of a housing and lumen, with a hypodermic needle affixed to the distal end of the lumen and recesses in the housing where the sensors(s) are connected. FIGS. 2A-2B show horizontal and vertical cross-sections of an embodiment of a housing and lumen. A hypodermic needle is affixed to the distal end of the lumen. The locations of sensors and electrical contacts, as well as a physical stop behind the connection area of the hypodermic needle, are shown in FIG. 2B. FIG. 3 shows an outer view of one embodiment of a housing and lumen. The distal end for connecting a hypodermic needle also includes a securing element, e.g., a flange, for connecting an item, e.g., a flexible cover, to the distal end of the inner housing. FIG. 4 shows an outer view of one embodiment of a housing and lumen, with a hypodermic needle affixed to the distal end of the lumen and an attachment element, e.g., a recess, in the inner housing for connecting to an outer housing.

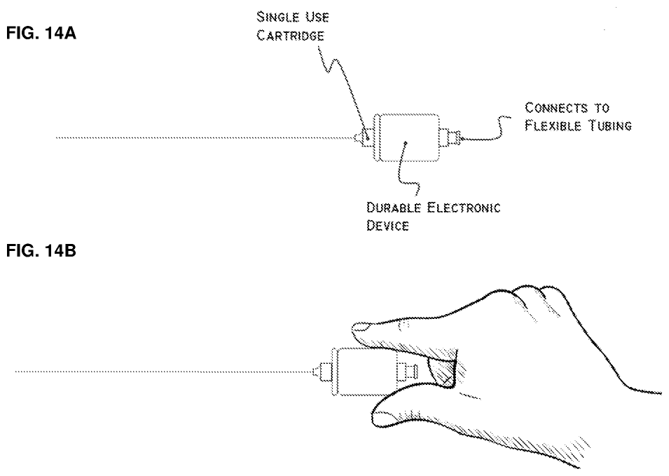

Devices of the invention may be configured to be small and portable such that they can be utilized by a clinician with a single hand. This is depicted in FIGS. 14A and 14B, which show a generalized embodiment of a device of the invention sized appropriately for grasping by a hand.

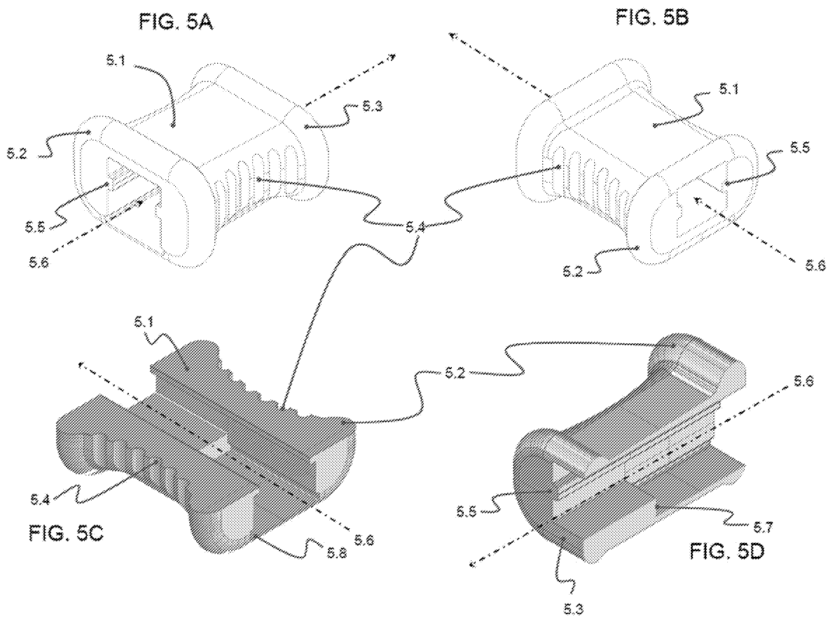

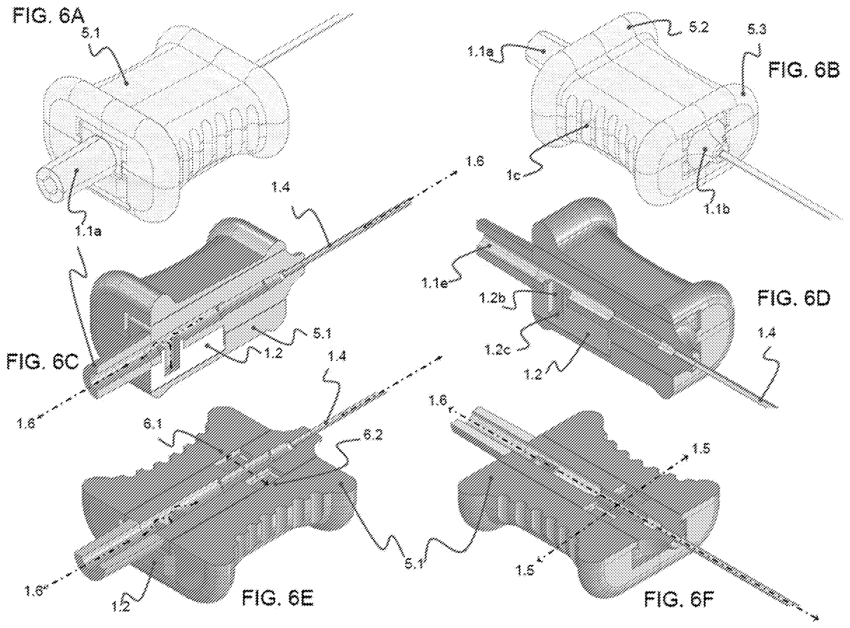

Devices may include a sensor and circuitry in the same or different housings. An embodiment of a housing that includes circuitry separate from the sensor is shown in FIGS. 5A-5D. FIGS. 5A-5D show outside (FIGS. 5A and 5B) and cross-section (FIGS. 5C and 5D) views of this housing. When multiple housings are employed, they may include mating features such as tongues, grooves, slots, stops, and tabs. Housings may be slideably engaged, e.g., where the housing with the fluid lumen slides wholly or partially within the other housing. Alternatively, the two housing may contact on one or more faces and have locking features. FIGS. 6A-6F, 7A-7D, 8A-8D, and 9 show embodiments of two housings slideably engaged. When more than one housing is employed, the connection can be permanent or releasable. When multiple housings are employed, one or both can be disposable. For example, the housing containing the circuitry can be reusable, while the housing containing the fluid lumen is disposable. Alternatively, the sensor and circuitry are in one housing that mates with a second housing including the lumen in a manner that operatively couples the sensor to the lumen.

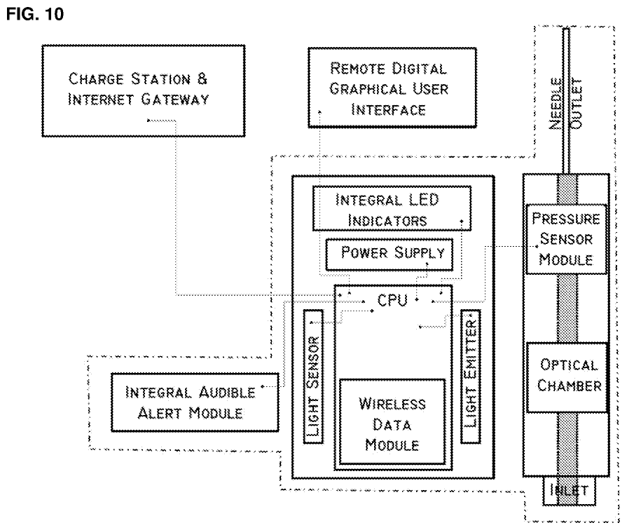

Devices may also be in communication with other components, e.g., for data storage, analysis, or output. Such other components can be connected to the device by wires or other electrical connection or wirelessly. A block diagram of an embodiment of the invention with electrical connections and data transmission components is shown in FIG. 10.

### Hypodermic Needles, Catheters, and Fluid Conducting Sources

Hypodermic needles (and lumens thereof) used for conducting fluids during medical procedures are either permanently affixed to the housing at the distal end of the lumen or releasably affixed, e.g., via a standard connector known in the art, such as a clamp, screw threads, bayonet, Luer lock, or Luer slip fitting. The needle can have any style known to those skilled in the art, such as any needle length, gauge, point profile, lancet style, primary/secondary bevel, anti-core heel, or needle coatings appropriate to the procedure.

In some cases, a hypodermic needle may have a catheter that circumferentially surrounds it. When in this configuration, the catheter may be deployed at the time of a procedure by advancing the catheter off of the hypodermic needle, thereby installing the catheter. The catheter remains in place to maintain access to the location, e.g., a peri-neural location, a tissue compartment, an intravascular compartment, where the hypodermic needle tip was placed after the hypodermic needle has been removed. In other embodiments, a catheter may be installed through a lumen, e.g., a lumen of a device of the invention, and through the hypodermic needle. In this configuration, the catheter will remain in its installed location when the device and hypodermic needle are removed. The catheter can be advanced through a proximal port of a device, or, alternatively, through an accessory, e.g., Y-type, port. Suitable catheters for use with a device of the invention are known in the art.

Fluid delivery into a device of the invention can be achieved through the use of delivery methods known in the art, e.g. operator pressure on a syringe barrel, syringe pump, or peristaltic pump. Fluid lines can be integral or attached to the proximal opening of a device of the invention through standard connectors known in the art, e.g., a clamp, screw threads, bayonet, Luer lock, or Luer slip fitting.

### Sensors

Sensors useful in the present invention include transducer type sensors, which convert a physical measurement (e.g. pressure or electrical, or optical properties) into an electrical signal. A sensor for the present invention is a pressure sensor capable of creating an electrical signal that is a function of the pressure. The signal resulting from the transducer can either be analog (DC) or digital, e.g., a digital pulse width modulation (PWM) signal or other encoded signal. Pressure transducers useful for a device of the present invention include, but are not limited to, inductive, resistive, piezoelectric, and capacitive transducers. Fluid content, e.g., blood, sensors useful for a device of the invention include optical sensors or electrical sensors. Other types of sensors, both pressure and fluid content, e.g., blood, are known in the art. In certain embodiments, the device includes both a pressure sensor and a fluid content, e.g., blood, sensor.

In certain embodiments, the pressure sensor includes a piezoelectric element. Different pressure ranges can be measured by choosing a sensor of appropriate material composition and physical size. All or portions of a pressure sensor can be isolated and protected from the fluids within the fluid conducting lumen by a dielectric gel. When the fluid in the lumen is subjected to a pressure differential (e.g., relative to ambient), that pressure is applied directly to the dielectric gel portion of the pressure sensor and thus to the active element of the sensor. Fluctuations in applied pressure result in corresponding variations in the data produced by the pressure sensor.

In certain embodiments, the fluid content, e.g., blood, sensor includes an optical sensor. Optical detection may occur by any suitable method, e.g., changes in absorbance, reflectance, fluorescence, turbidity, or scattering. Typically, the sensor includes a light source, e.g., an LED, and a detector, e.g., a photodiode. The light source can be a broadband source or be a single frequency. The light source and detector can be arranged across the lumen from one another, on the same side of the lumen, or in any other geometry suitable for detection of light. In certain embodiments, the blood sensor includes a plurality of light detectors and/or sources.

In other embodiments, the fluid content, e.g., blood, sensor includes an electrical sensor. Electrical detection may occur by any suitable method, e.g., changes in resistance, impedance, inductance, or capacitance. Suitable sensors, e.g., electrodes, for electrical detection are known in the art.

The device may also include a gyroscope, accelerometer, or combination thereof. Such components may be in used to track overall movement of the device and rate of movement. When multiple housings are present, such components may be placed in the outer or inner housing, in particular the outer housing.

### Circuitry

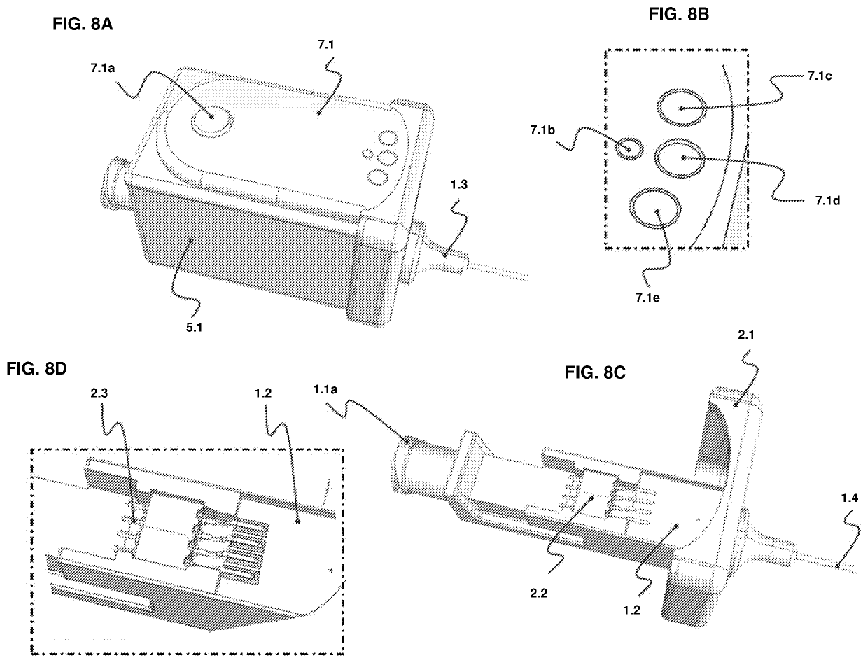

The invention employs circuitry to process data produced by the sensor and provide an indicator signal of the value. The circuitry may be contained in the same housing as the sensor, or the device may include an outer housing that mates with the housing containing the sensor. When two housings are employed, they will be connectable to allow for signal transfer from the sensor to the circuitry. Typically, this connection will be a direct electrical connection, but other connections, such as optical, RF, or other wireless connection may be employed. The circuit may be a microprocessor-based device with programming configured to take as input the voltages, e.g., analog or digital, such as PWM, sent from the sensors and to output an indicator to the user. Alternatively, simpler electronics may be employed such as a voltage comparator. Components for the circuitry include, but are not limited to, operational amplifiers, power supplies, capacitors, ADC/DAC, and flow rate counters. The circuit is designed to provide an indicator signal to the user based on the output of the sensors measuring the pressure and/or fluid content, e.g., presence or amount of blood. The indicator signal in turn is used to produce an indicator of the pressure or fluid content, e.g., presence or amount of blood. This indicator can be audible (e.g., a tone, beep, or pre-recorded speech), visual (e.g., flashing/blinking light or display with text), or tactile (e.g., vibration), or another sensory modality). For example, FIGS. 8A, 8B, 9, and 10 show the locations of indicator lights on the exterior of various embodiments of devices according to the invention. The indicator may further be indicative of a threshold by providing a binary yes or no indicator, or a categorical outcome measure, or a mathematical probability of a related event. The threshold may vary depending on the specific application of the device. Alternatively, the indicator may be indicative of an absolute or relative value within a range. The indicator may be part of the device, or the indicator signal may be passed to another device, e.g., a computer display or monitor, speakers or headphones, or any other output device, to produce the indicator. The indicator signal may be in a digital or analog form that may be stored for later retrieval. In addition to the above, the indicator signal can provide a probability statistic given the history of the data stored within the circuitry rather than providing a simple binary or categorical decision determined by exceeding a pre-set threshold value. The device may also contain or be in communication with a storage medium, e.g., flash memory, or a data server.

In certain embodiments, the circuitry contains an operational amplifier configured to act as a voltage comparator. The voltage comparator can be preset with a threshold voltage on one of its input terminals with the other input terminal connected to the output of a sensor. The output of the voltage comparator determines whether an indicator is delivered to the user based on standard programming binary logic gates, e.g., AND, OR, NAND, NOR, XOR, XNOR, and inverter/NOT.

In certain embodiments, the circuitry of a device of the invention can also include a pre-programmed microcontroller which can accept the output voltage, e.g., analog or digital, such as PWM, from a sensor.

When an indicator is triggered, the circuitry can be reset manually or automatically to clear the indicator. For example, when the pressure or fluid content, e.g., blood, sensor no longer registers a value that triggers the indicator, the indicator may discontinue. Alternatively, the indicator may discontinue after a set period of time, e.g., after the trigger condition has been removed, or only after a manual reset.

The device may also allow for suppression of false positives, e.g., caused by air bubbles. The lens effect of the meniscus at the leading and/or trailing edges of an air bubble results in significant reflection and refraction and rapid fluctuations in the levels of light incident upon the photodiode light sensor per unit of time. These fluctuations can cause the voltage output of the photodiode to rapidly cycle above and below the alarm threshold voltage, resulting in “false positive” alarm conditions. The output, e.g., VDC/PWM output of a photodiode sensor, may therefore be fed into the circuitry, e.g., a microcontroller unit (MCU) component of an integrated circuit (IC), that identifies air bubble events and suppress their impact on the circuitry or that employs signal filtering to account for artifacts such as air bubbles. For example, the MCU can be configured such that fluctuations of the VDC/PWM output must be sustained over a predetermined time interval in order to activate the alarm circuit. In another embodiment, the device may measure absorption of specific wavelengths of light energy by the fluid, e.g., containing blood, as a means of reducing false positive alarm states. The circuitry, e.g., MCU, can be configured such that it compares relative ratios of light energy absorption at different wavelengths. For example, blood has a higher degree of absorption in the range of visible light close to red light (approx. 700 nm) compared with blue light (approx. 400 nm). The near infrared (NIR) spectrum may also be utilized.

### Methods of Use

The invention features methods for measuring fluid properties, i.e., pressure and/or fluid content, e.g., presence or amount of blood, during a medical procedure, e.g., an injection. The devices may be employed in any procedure sensitive to the placement of the needle or pressure of injection or aspiration. The methods may be used to determine whether a needle is placed in the appropriate location or when the needle is placed is an inappropriate location. For example, devices employing a fluid content sensor, e.g., blood, sensor can be used to ensure that the needle is outside of or inside of a blood vessel or other compartment. In addition, devices employing a pressure sensor can be used to determine the location of the needle, i.e., within a tissue having a pressure above or below a threshold, or to determine proper introduction or removal of fluid, e.g., to ensure that pressure is at an appropriate level during a procedure or to determine if a blockage or partial blockage has occurred. An exemplary use of the devices of the invention is in pediatric or adult anesthetic procedures, such PNB, epidural nerve block, or dental anesthesia.

An exemplary use of the invention is in determining proper placement of a needle during a PNB. Opening injection pressure (OIP) is a concept used to distinguish the hydraulic pressure in the hypodermic needle, syringe, and tubing resulting from the variable force applied to the syringe plunger by the user, from the pressure that results from the needle tip being located in a tissue compartment with relatively low compliance. Proper needle tip location during injection procedures is commonly between two different tissue types (e.g., between nerve and muscle or fascia). The compliance of such a “tissue plane” is relatively high in comparison to direct injection within a particular tissue and allows the clinician to avoid excessive OIP. For example, OIP values of greater than 12 psi have been associated with needle tip placement within a nerve fiber, resulting in nerve injury from direct laceration of nerve fibers via the needle, neural ischemia from excess pressure created by the injected local anesthetic, or direct chemical toxicity from the anesthetic itself. Accordingly, devices of the present invention can be used to determine the OIP for a procedure to ensure that the needle is not within a nerve fiber. The output of the pressure sensor is fed into the circuitry, e.g., one of the input terminals of an operational amplifier voltage comparator with a pre-determined threshold voltage programmed into the circuitry is fed into the second input of the operational amplifier voltage comparator. If the pressure measured by the sensor falls below that of the pre-determined threshold, the programmed indicator is not triggered, and the clinician can elect to proceed with the nerve block. If the pressure measured by the sensor exceeds that of the predetermined threshold, e.g., 12 psi or 15 psi, the programmed indicator is activated to alert the user of the high pressure, indicating that the needle has contacted a nerve fiber and needs to be repositioned before continuing with the nerve block.

In some cases, the pressure measured is a pressure waveform, e.g., a pulsatile pressure waveform or vascular pressure waveform, which may be used to confirm the location of a hypodermic needle within a cavity, e.g., the epidural space. For example, the measurement of venous or arterial pressure waveforms can be used to confirm location within a central vein or an artery prior to cannulation with a catheter. In addition, a combination of pressure measurement may be utilized to confirm the position of a needle during a procedure. For example, a combination measurement of OIP and a vascular pressure waveform, e.g., a venous or arterial pressure waveform, may also be used to confirm the position of a needle during a procedure.

The invention can also determine whether the needle is placed in a blood vessel by aspiration. Aspiration is achieved when a negative pressure, e.g. suction, is applied to a syringe or other fluid delivery system during a procedure such as a PNB. A “positive” aspiration event is said to have occurred when blood is present within the fluid. In some procedures, e.g., diagnostic interrogations or therapeutic interventions, a positive aspiration event is a confirmation of successful vascular access. In other instances, a positive aspiration is an indication of unintended vascular access, alerting the clinician that the needle tip must be withdrawn and repositioned before the procedure can continue. Accordingly, the devices of the invention can be used to determine whether or not the needle is in a blood vessel. The fluid content, e.g., blood, sensor in a device can measure fluid aspirated in the lumen, e.g., by optical or electrical methods. In one example, light from a light source is directed through the aspirated fluid, and light passing through the fluid is collected on a light detector. The light detector can return a specific DC voltage or other output based on the incident flux on the light detector's active element. An operational amplifier may be configured to receive the voltage from the light detector as one of its inputs. The second input voltage to the voltage comparator is pre-determined from the light detector's output voltage corresponding to a fluid to be injected, e.g., a clear, colorless fluid. During aspiration concurrent with unintended vascular access, blood from the tissue is sampled and drawn into the lumen and inner housing. When the emitter encounters blood rather than a clear fluid, a greater portion of the light energy emitted is absorbed by the blood than was absorbed by the fluid. In this example, the amount of light energy that reaches the light detector is correspondingly less, resulting in a lower output voltage from the light detector, and an indicator is triggered as the lower voltage is below that of the threshold set by the output when the fluid contains no aspirated blood. The indicator condition can persist until such time as the blood is cleared and the relative magnitude of the voltage comparator inputs revert.

### Flexible Cover

Maintaining sterility is paramount for ensuring patient safety and adhering to regulations. Thus, any medical device that cannot be sterilized by existing sterilization techniques must be enclosed in a sterile barrier for entry into a sterile field. An ideal sterile barrier completely seals the device such that no microorganisms can pass from the non-sterile device to the sterile field. This barrier can be applied in a manner that maintains the sterility of the clinician and the exterior of the barrier.

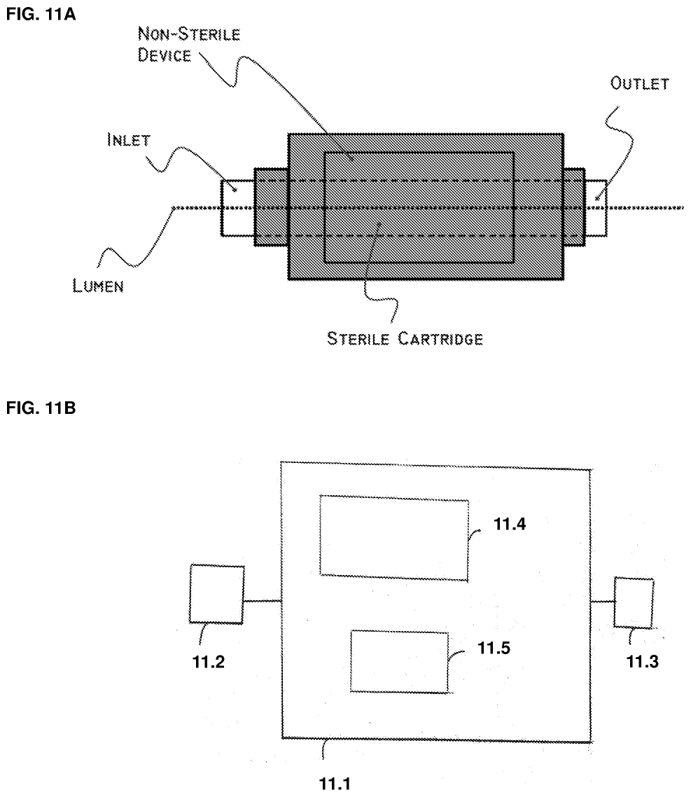

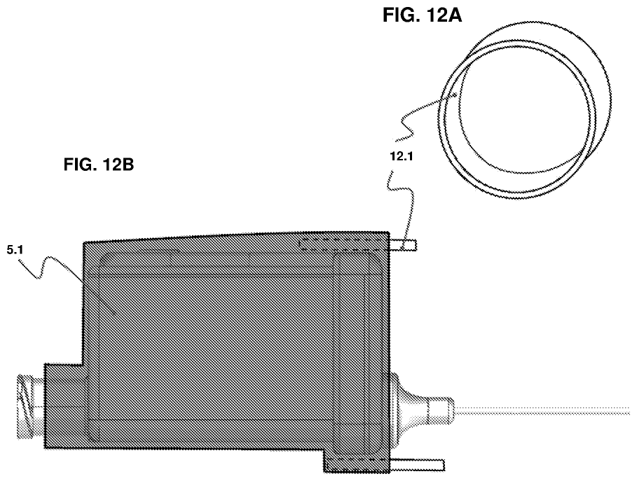

Accordingly, the invention includes a flexible cover for covering a structure, e.g., a medical device of the present invention. The flexible cover is typically constrained prior to use. The flexible cover can be constrained by any method where the volume of the cover is reduced, e.g., by folding, compression, rolling, or a combination thereof. A flexible cover may be connected, e.g., releasably, to an application assistance structure such that the two can be separated after the flexible cover is placed on the structure. When installed and the application assistance structure removed, the flexible cover can assume substantially the same shape as the structure that it is covering. An example of a cover is presented in FIGS. 11A-11B, in which FIG. 11A shows a block diagram of a medical device including both a sterile inner portion and a non-sterile outer portion. FIG. 11B depicts a block diagram of an embodiment of a flexible cover enclosing a medical device. The flexible cover (11.1) encloses multiple structures (11.4, 11.5). In the figure, 11.4 can represent a non-sterile, medical device containing, but not limited to, a printed circuit board, an internal power source, and various sensor systems, and 11.5 can represent a sterile, disposable medical device that integrates with 11.4 in such a way that the sensor systems in 11.4 interact with 11.5. The pass through channel, e.g., a lumen, is located in 11.5. The flexible cover contains two points of access (11.2, 11.3) that allow for the passage of sterile substances through the assembled and covered device. Here, 11.2 represents the distal (closer to the patient) end of the assembly and 11.3 represents the proximal (closer to the clinician) end of the assembled device. These points of access may be at any two distinct locations on the medical device. FIGS. 12A-12B show side view and top-down views of an embodiment of the apparatus with an annular application assistance structure, e.g., a ring, installed at the edge of a structure, e.g., a medical device.

In certain embodiments, the flexible cover may be sterile, e.g., made of a material that can be sterilized. For example, the flexible cover, e.g., the outside surface of the flexible cover, may be sterile before application to a structure. Alternatively, the outside surface of the flexible cover may be sterilized after application to a structure, e.g., chemically. Additionally, the inside surface of the flexible cover may be sterile. Sterilization can occur by methods known in the art, including, but not limited to, exposure to radiation, heat, steam, ethylene oxide gas, hydrogen peroxide gas, or electron beams.

In addition to being sterilizable, the material used may be biocompatible, impenetrable by microorganisms, e.g., viruses and bacteria, durable, and sufficiently flexible such that it can provide the proper fit around the structure to be covered but also sufficiently rigid such that it can be shaped. In general, the flexible cover has the approximate shape of the structure it will cover. Suitable materials for the flexible cover includes polymers, including, but not limited to, silicones, urethane, e.g., polyurethane, polytetrafluoroethylene (PTFE), expanded PTFE (ePTFE), fluorinated ethylene propylene (FEP), perfluoroalkoxy (PFA), ethylene tetrafluoroethylene (ETFE), polyvinylidene fluoride (PVDF), polyethylene, and polypropylene. An exemplary material for the flexible cover is silicone. In some cases, the material has sufficient transmissive properties, e.g., optically transparent, so that it does not interfere with any indicators, e.g., LEDs, of the structure, e.g., a medical device.

In some cases, the flexible cover is a disposable, e.g., single use, item. Alternatively, the flexible cover can be a reusable item that can be sterilized prior to each administration.

Medical devices, such as those of the present invention, may be used to administer fluids, drugs, or other medical substances, or to introduce surgical or interventional devices and/or catheters into a patient. In some embodiments, the medical device requires a passage through the interior of the device, e.g., a lumen. The flexible covers have at least one opening. For example, a flexible cover that is configured to cover a device that delivers a therapeutic fluid from a fluid conducting source, e.g., a syringe, through a hypodermic needle lumen, may have two access ports. These access ports may be sized to allow standard connections, including, but not limited to, clamp, screw threads, bayonet, Luer lock, or Luer slip fitting, on the medical device to pass through the access ports so connections can be made. Alternatively, access to the covered structure may also occur through puncturing the material with a suitable device, e.g., a hypodermic needle.

In some cases, the distal end of a structure, e.g., a medical device, may have a securing element, e.g., a flange, that engages the distal end of the flexible cover and secures it to the medical device. Alternatively, the flexible cover can be secured to the medical device using an external fastener, e.g., a sterile adhesive band or a clamp. In addition, the securing feature may be the flexible cover itself. For example, the fit between the flexible cover and the structure can be such that the flexible cover is held tightly against the structure. Other securing methods are known in the art.

### Application Assistance Structure

An application assistance structure of the invention includes a structure, e.g., rigid, that is connected, e.g., releasably, to a constrained flexible cover. The application assistance structure allows the constrained flexible cover to be deployed e.g., to assume substantially the same shape as the structure being covered. The application assistance structure consists of a rigid outer structure having an opening that is sufficiently large such that, when the constrained flexible cover is connected, the structure to be covered, e.g., a medical device, can pass freely through the opening.

The application assistance structure may be of any suitable shape, e.g., annular or polygonal, e.g. triangular, diamond, or hexagonal. The application assistance structure may have a similar shape as the structure to be covered; alternatively, the application assistance structure may be of a different shape. FIG. 12A provides an example of an application assistance structure that is annular, e.g., a ring, used to assist in applying a constrained flexible cover to a rectangular-shaped structure. It will be appreciated by the skilled artisan that any polygonal shape may be used for the application assistance structure.

The application assistance structure may be made of any suitable material to allow a clinician to grasp when applying the constrained flexible cover to the structure. In certain embodiments, the application assistance structure may be sterile, or made of a material that can be sterilized. Suitable materials for the application assistance structure include, but are not limited to, metals (e.g., stainless steel or aluminum), ceramics, polymers (e.g., polyethylene terephthalate (PET) or polyoxymethylene), or glass. Alternatively, a portion of the application assistance structure can be sterile, e.g., a portion that contacts the constrained flexible cover.

### Methods of Application

The invention features methods for applying a cover, e.g., a constrained flexible cover, to a structure, e.g., a medical device. The flexible cover may be employed to isolate a medical device that is not sterilized. An exemplary use of the flexible cover is for isolating the body of a pass through device for introducing therapeutic fluids in pediatric or adult anesthetic procedures, such as PNB, epidural nerve block, or dental anesthesia.

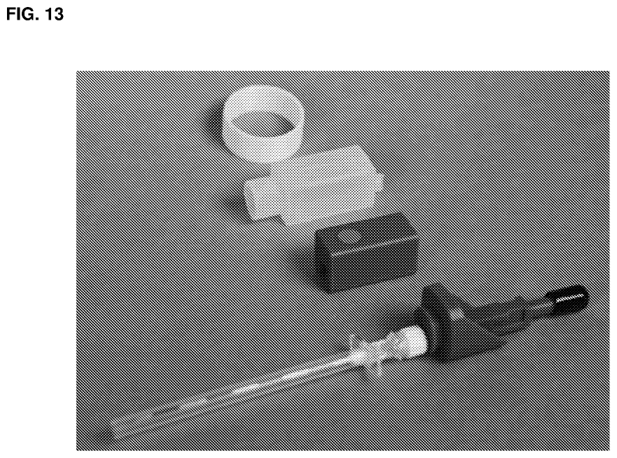

In some embodiments, a proximal end of the structure, e.g., the end of a medical device closest to the clinician, to be covered with the flexible cover is inserted into the opening of an application assistance structure, to which the constrained flexible cover is connected. The constrained flexible cover can then be deployed by moving the application assistance structure from the proximal end of the structure to the distal end of the structure, thereby allowing the constrained flexible cover to be extended over the distal end of the structure, covering its surface. FIG. 13 shows an embodiment of a complete apparatus and medical device to be covered using a silicone cover substantially the same shape as the medical device and an annular application assistance structure. In some cases, one of the constrained flexible cover or the application assistance structure, or both, are sterile prior to application to the structure.

### EXAMPLES

### Example 1—Inner and Outer Housings

FIGS. 1A-1D shows an example of an inner housing intended to be a single use consumable product. Alignment grooves (1.1d) allow for a proper fit within an outer housing. The needle cartridge body contains a Luer slip fitting (1.1a) for attaching a fluid source. A needle (1.4) is attached to the cartridge by seal (1.3), and the body includes a tapered nose cone (1.1b) around the needle connection. The needle cartridge contains a piezoresistive pressure sensor (1.2) for measuring the pressure of the fluid within the needle cartridge, interfacing with the outer housing via a connectivity plate (1.2a). The sensor includes a sensing element which is separated from the injection lumen by a dielectric gel (1.2c). The piezoresistive pressure sensor is sealed to the body at port 1.2d. The connectivity plate (1.2a) has alignment features that allow it to mate with a corresponding alignment feature on an outer housing. The cross-section views in FIGS. 1C, 1D, and 2B indicate the action of piezoresistive pressure sensor more clearly. Fluid is injected through the lumens of the needle cartridge and hypodermic needle along the fluid flow axis (1.6), first into the main body lumen (1.1e) then through the exit of the cartridge (1.1g). As the fluid flows through lumens of the needle cartridge, it fills a small chamber (1.2b) containing a piezoresistive pressure sensor (1.2) and a dielectric gel (1.2c), which measures the pressure of the fluid and produces an indicator signal.

The needle cartridge also has an optical sensing region (1.1c) where optical sensors, e.g., for the detection of blood during aspiration, are located. Fluid is aspirated back in to the needle cartridge, filling a chamber (1.1f) just behind the hypodermic needle. Light is emitted from an optical emitter, transmitted through the liquid, and the transmitted light is collected on an optical collector, with the beam path of the optical emitter (1.5) perpendicular to the flow of liquid through the lumen of the needle cartridge.

Another embodiment of an inner housing according to the invention is shown in FIGS. 2A-2B. This example contains an externally molded stop (2.1) that prevents the inner housing from sliding through an outer housing; the stop has a curve that mates with the outer housing. The externally molded stop (2.1) and its curve ensure a solid electrical connection between the inner housing's electrical components, e.g., sensor(s), and the circuitry of an outer housing. For example, the electrical components of the inner housing include a spring pin header (2.2) and solder tails (2.3) connected to the piezoresistive pressure sensor (1.2), which sits above a reservoir for dielectric gel (1.2c), as shown in FIGS. 2A-2B.

An outer view of another embodiment of an inner housing and a lumen according to the invention is shown in FIG. 3. In the embodiment of FIG. 3, the distal end for connecting a hypodermic needle also includes a securing element, e.g., a flange, for connecting a cover to the distal end of the inner housing.

An isometric view of another embodiment of an inner housing and a lumen according to the invention is shown in FIG. 4. The inner housing shown in FIG. 4 is rectangular in shape and includes a hypodermic needle affixed to the distal end of the inner housing, a proximal opening for connecting a source of fluid, and further includes an attachment element, e.g., a recess, in the inner housing for connecting to an outer housing.

FIGS. 5A-5D show an embodiment according to the invention of a reusable outer housing containing the circuitry of the device. An inner housing (1.1) is designed to slideably engage the outer housing (5.1) along the insertion axis (5.6), providing electrical connections. Alignment tongues (5.5 and 5.8) in the outer housing provide proper alignment for an inner housing (1.1) upon insertion. The outer housing (5.1) also includes an internal stop (5.7) to prevent the inner housing (1.1) from sliding through the outer housing (5.1). The outside edges (5.8) of the inner opening of the outer housing (5.1) are an alignment feature that allow it to mate with a corresponding alignment feature of an inner housing, e.g., the connectivity plate (1.2a) shown in FIGS. 1A and 1C.

As shown in FIGS. 5A-5D, the overall shape of the outer housing (5.1) may be tapered such that it can be comfortably held in a single hand by a clinician. The ends of the outer housing in this embodiment are flared (5.2 and 5.3) and terminate in rings to provide the clinician with a surface for improved grip and thus more control when employing the device. The central body (5.1) of the outer housing contains grip enhancing elements (5.4) shaped into the body of the outer housing.

### Example 2—Assembled Devices

FIGS. 6A-6F show various views of an assembled device according to the invention, where the needle cartridge (1.1) has been inserted into a tapered outer housing (5.1). The piezoresistive pressure sensor (1.2) fits against the outer housing as seen in FIGS. 6C and 6D. For a measurement of the optical properties of fluid using the complete device, fluid is aspirated back into the device and into the beam path (1.5) of a light emitter (6.1) and a light collector (6.2) mounted within the housing.

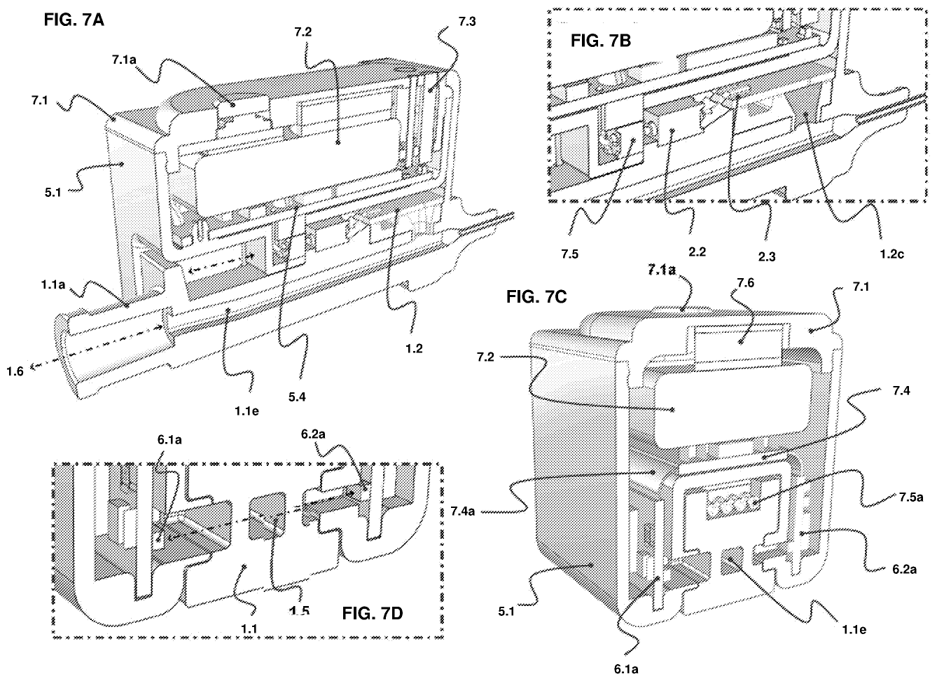

FIGS. 7A-7D show an alternate embodiment of an assembled device according to the invention including an outer housing (5.1) having a non-tapered rectangular shape with an opening for the inner housing. The top of the outer housing has a removable cap (7.1) that contains a button (7.1a) for powering on the device. The cap can be removed from the outer housing to access a compartment that contains a battery (7.2) and light pipes (7.3) to display indicator lights. The horizontal cross-section shown in FIG. 7B provides a zoomed in view of the electrical connections (such as the spring pin header (2.2), solder tails (2.3) connected to the piezoresistive pressure sensor (1.2), and the target header (7.5) for accepting the spring pins of the spring pin header (2.2)) between the inner and outer housings as well the piezoresistive pressure sensor (1.2) and a dielectric gel (1.2c), which measures the pressure of the fluid, producing an indicator signal. The vertical cross-section in FIGS. 7C and 7D show the locations of the fluid lumen (1.1e), battery (7.2), circuit boards for the light emitter (6.1a) and a light collector (6.2a), the main CPU board (7.4), ribbon connectors (7.4a) to connect the circuit boards for the light emitter (6.1a) and light collector (6.2a) to the CPU board (7.4), electrical pin connectors to the target header connectors (7.5a), and charge antenna (7.6). FIG. 7D shows the zoomed in view of the optical path (1.5) of the joined inner and outer housings, indicating the optical path (1.5a) of a light emitter (6.1) and a light collector (6.2a) mounted within the outer housing.

As shown in FIGS. 8A-8B, an alternate embodiment of the outer housing (5.1) has a non-tapered rectangular shape that has a lid (7.1) having both a power button (7.1a) and indicator lights (7.1b, 7.1c, 7.1d, and 7.1e shown in the zoomed in view of FIG. 8B). The indicator lights can be grouped into two different groups, each group having a different function. Indicator light 7.1b corresponds to the power indicator, indicating that the device has sufficient battery power for operation. The indicator lights 7.1c, 7.1d, and 7.1e correspond to runtime status indicators, and are indicative of whether the device is ready for data acquisition (7.1c), the device in standby mode, currently acquiring data, or is approaching an alert or alarm threshold (7.1d), or has an active alert or alarm (7.1e).

FIGS. 8C and 8D show outer (FIG. 8C) and zoomed in (FIG. 8D) views of an alternate embodiment of the inner housing providing the layout of the piezoresistive pressure sensor (1.2) and electrical connections, e.g., spring pin header (2.2) and its incorporated solder tails (2.3) of the piezoresistive pressure sensor (1.2), of the inner housing.

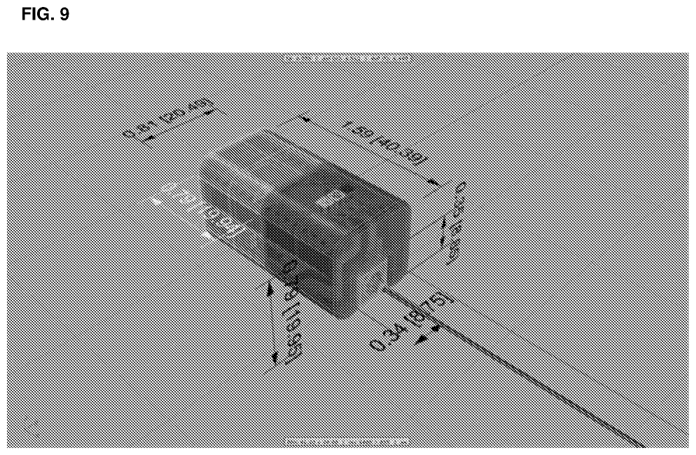

FIG. 9 shows an embodiment of a complete device of the invention with the inner housing, affixed with a hypodermic needle at its distal end, inserted into the outer housing. The housing has three indicator lights on the top of the device above the connection for the hypodermic needle.

FIG. 10 shows a block diagram of an embodiment of the invention including the medical device, e.g. the inner housing with a lumen and both optical and pressure sensing modules and an outer housing including CPU, light emitter and photodiode, power supply, indicator lights, and an audible indication system. FIG. 10 also shows additional components of a system for use within the device, including a charge station to recharge the on-board battery, an internet gateway for sending acquired data, and a graphical user interface (GUI) so the clinician can visualize the results of acquisition, e.g., receiving in substantially real time, and communicate procedure guidance to the clinician.

### Example 3—A Flexible Cover for Isolating a Non-Sterile Medical Device

FIGS. 12A, 12B, and 13 show an exemplary flexible cover. In FIG. 13, the flexible cover has the approximate shape of the medical device to provide a tight fit, with an access port on both ends. Specifically, the flexible cover is configured to cover the outer housing of a device of the present invention having a rectangular shape with rounded edges. The cover seals around the device at the proximal and distal ends.

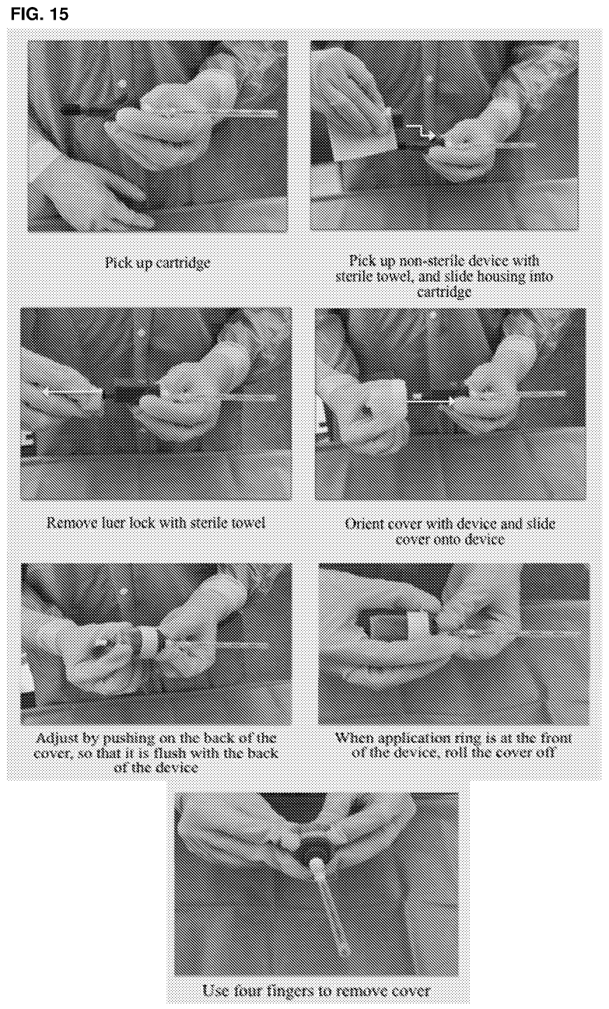

As shown in FIG. 15, to apply the cover, a non-sterile portion of the medical device, e.g., an outer housing comprising sensor circuits, e.g., fluid content, e.g., blood, and/or pressure sensor circuits, is grasped with a non-sterile hand or, alternatively, with a sterile towel over a sterile gloved hand. A sterile portion of a medical device, e.g., an inner housing with a hypodermic needle, is grasped with a sterile gloved hand. The sterile portion of the medical device is inserted into the non-sterile portion. Portions of the sterile portion protrude through the proximal, e.g., towards the clinician, and distal, e.g., towards the subject, ends of the cover. To facilitate handling, a protruding region of the sterile portion of the medical device may be covered with a removable cap to prevent contamination by the insertion of the sterile portion into the medical device. If such caps are utilized, they can be removed by the non-sterile hand.

The clinician, using a sterile gloved hand, grasps the constrained flexible cover (including an application assistance structure releasably connected thereto). In this example, the medical device is supported by the clinician via the sterile portion of the medical device. Holding the medical device with the sterile portion inserted, the clinician inserts one end of the medical device, e.g., the proximal end, into the constrained flexible cover and uses the application assistance structure to push the cover over the medical device, e.g., from the proximal end of the medical device to the distal end of the medical device. The application assistance structure can then be removed. The deployed flexible cover can then be secured to medical device.

### Example 4—Optical Output from a Device of the Invention

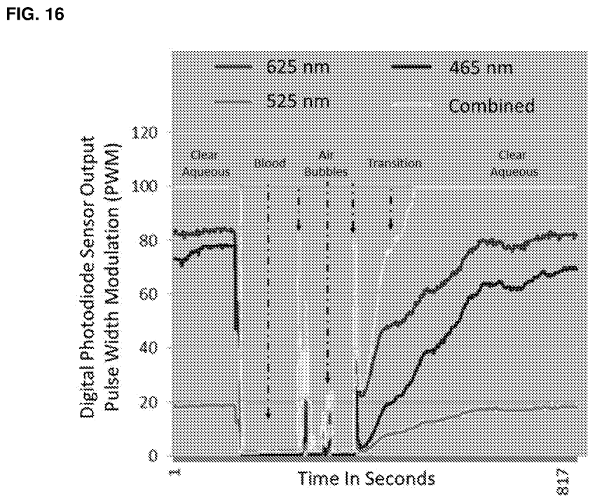

FIG. 16 shows an example of typical output data of an embodiment of a device according to the invention having a fluid content, e.g., blood, sensor to measure absorbance of light at a photodiode. In this example, a test injection procedure was simulated by first introducing a clear aqueous anesthetic under pressure. At specific time intervals, blood and air bubbles were introduced to simulate the aspiration of a syringe during an injection procedure, where a bodily fluid would be introduced into the optical path of a device according to the invention.

The output signal from the device according to the invention is a digital pulse width modulation (PWM) signal for multiple wavelengths of light emitted from the light emitter. The data in FIG. 16 correspond to a single time series. The events where blood crossed into the optical path of the device are evident by the dramatic reduction in photodiode output, i.e., blood present at the optical gate will absorb the vast majority of light, independent of wavelength. Thus, less light reaches the photodiode light sensor, and the photodiode output drops accordingly. Air bubbles that are introduced into the optical path result in a reduction of photodiode output as well; however, the response is stochastic, e.g., highly variable, in comparison to the blood-present condition, but also serves to reduce the output of the photodiode.

Other embodiments are in the claims.

What is claimed is:

1. A device for monitoring fluid properties, the device comprising:
a) a housing comprising a first lumen with a proximal opening and a distal opening;
b) a hypodermic needle having a second lumen permanently affixed to the housing so that the first and second lumens are fluidically connected via the distal opening; and
c) a pressure sensor operatively coupled to the first or second lumen to measure fluid pressure in the first or second lumen.

2. The device of claim 1, wherein the hypodermic needle further comprises a catheter releasably connected the needle.

3. The device of claim 1, further comprising a fluid conducting source fluidically connected to the proximal opening of the first lumen.

4. The device of claim 1, further comprising circuitry configured to process data collected by the pressure sensor and provide an indicator signal thereof.

5. The device of claim 1, further comprising circuitry configured to collect, store, and/or transmit data collected by the pressure sensor.

6. The device of any of claims 1-5, wherein the pressure sensor is an inductive, resistive, piezoelectric, or capacitive transducer.

7. The device of any of claims 1-6, wherein the pressure sensor is a piezoelectric transducer.

8. The device of claim 4, further comprising an indicator actuated by the indicator signal.

9. The device of any of claims 1-8, further comprising a fluid content sensor operatively coupled to the lumen.

10. The device of claim 9, wherein the fluid content sensor comprises an optical sensor.

11. The device of claim 10, wherein the optical sensor comprises a light detector and a light source.

12. The device of claim 9, wherein the fluid content sensor comprises an electrical sensor.

13. The device of claim 9, wherein the fluid content sensor is a blood sensor operatively coupled to the lumen to determine the presence or amount of blood in the lumen.

14. The device of claim 8, wherein the indicator is an audible, visual, or tactile indicator.

15. The device of claim 4, wherein the device transmits the indicator signal externally.

16. A device for monitoring fluid properties, the device comprising:
a) a housing comprising a lumen with a proximal opening and distal opening; and
b) a fluid content sensor operatively coupled to the lumen.

17. The device of claim 16, wherein the fluid content sensor is a blood sensor operatively coupled to the lumen to determine the presence or amount of blood in the lumen.

18. The device of claim 16, further comprising a hypodermic needle having a second lumen so that the first and second lumens are fluidically connected via the distal opening.

19. The device of claim 17, wherein the hypodermic needle further comprises a catheter releasably connected to the needle.

20. The device of claim 17, wherein the hypodermic needle is permanently affixed to the housing.

21. The device of claim 17, wherein the hypodermic needle is releasably affixed to the housing.

22. The device of any of claims 16-21, wherein the fluid content sensor comprises an optical sensor.

23. The device of claim 22, wherein the optical sensor comprises a light detector and a light source.

24. The device of any of claims 16-21, wherein the fluid content sensor comprises an electrical sensor.

25. The device of any of claims 16-24, further comprising circuitry configured to process data collected from the fluid content sensor and provide an indicator signal thereof.

26. The device of any of claims 16-24, further comprising circuitry configured to collect, store, and/or transmit data collected by the fluid content sensor.

27. The device of claim 25, further comprising an indicator actuated by the indicator signal.

28. The device of any of claims 16-27, further comprising a pressure sensor operatively coupled to the lumen to measure fluid pressure in the lumen.

29. The device of claim 28, wherein the pressure sensor is an inductive, resistive, piezoelectric, or capacitive transducer.

30. The device of claim 28, wherein the pressure sensor is a piezoelectric transducer.

31. The device of any of claims 28-30, further comprising circuitry configured to process data collected by the pressure sensor and provide a pressure indicator signal thereof.

32. The device of claim 31, further comprising a pressure indicator actuated by the pressure indicator signal.

33. The device of claim 25, wherein the device transmits the indicator signal externally.

34. The device of any of claims 16-33, further comprising a fluid conducting source fluidically connected to the proximal opening of the first lumen.

35. A kit comprising:
a) an inner housing comprising a first lumen with a proximal opening and distal opening and a pressure sensor operatively connected to the first lumen to measure fluid pressure in the first lumen or a fluid content sensor operatively connected to the first lumen; and
b) an outer housing containing circuitry electrically configured to process data collected by the pressure or blood sensor and provide an indicator signal thereof,
wherein the inner housing mates with the outer housing to provide connectivity between the pressure or fluid content sensor and the circuitry.

36. The kit of claim 35, wherein the fluid content sensor is a blood sensor operatively connected to the first lumen to measure the presence or amount of blood in the first lumen.

37. The kit of claim 35, wherein the inner housing further comprises a hypodermic needle having a second lumen so that the first and second lumens are fluidically connected via the distal opening.

38. The kit of claim 35, further comprising a hypodermic needle having a second lumen and being connectable to the first lumen via the distal opening.

39. The kit of claim 37 or 38, wherein the hypodermic needle further comprises a catheter releasably connected to the needle.

40. The kit of any of claims 35-39, wherein the inner housing comprises both the pressure sensor and the fluid content sensor.

41. The kit of any of claims 35-40, wherein the fluid content sensor comprises an optical sensor.

42. The kit of claim 41, wherein the optical sensor comprises a light detector and a light source.

43. The kit of any of claims 35-40, wherein the fluid content sensor comprises an electrical sensor.

44. The kit of any of claims 35-43, wherein the inner or outer housing further comprises an indicator actuated by the indicator signal.

45. The kit of claim 44, wherein the indicator comprises a visual, tactile, and/or audible indicator.

46. The kit of any of claims 35-45, wherein the circuitry transmits the indicator signal externally.

47. The kit of any one of claims 35-46, wherein the circuitry is configured to collect, store, and/or transmit data collected by the pressure or fluid content sensor.

48. The kit of any of claims 35-47, wherein the pressure sensor is an inductive, resistive, piezoelectric, or capacitive transducer.

49. The kit of claim 48, wherein the pressure sensor is a piezoelectric transducer.

50. The kit of any of claims 35-49, further comprising a fluid conducting source fluidically connectable to the proximal opening of the first lumen.

51. A method for measuring pressure during a medical procedure, the method comprising:
a) providing a device comprising a housing comprising a first lumen with a proximal opening and a distal opening; a hypodermic needle having a second lumen attached to the housing so that the first and second lumens are fluidically connected via the distal opening; a pressure sensor operatively coupled to the first or second lumen to measure fluid pressure in the first or second lumen; and circuitry configured to process data collected by the pressure sensor and provide an indicator thereof;
b) inserting the hypodermic needle into a site of the medical procedure; and
c) measuring the pressure in the first or second lumen during the medical procedure via the pressure sensor to produce data,
wherein the circuitry analyzes the data and generates an indicator signal representative of the pressure.

52. The method of claim 51 wherein the indicator signal is generated when the pressure is above or below a threshold pressure.

53. The method of claim 51, wherein the pressure measured is the opening injection pressure.

54. The method of claim 51, wherein the pressure measured is from a pulsatile pressure waveform.

55. The method of claim 51, wherein the pressure measured is from a vascular pressure waveform.

56. The method of claim 51, wherein the medical procedure is a peripheral nerve block.

57. A method for the detection of blood during a medical procedure, the method comprising:
a) providing a device comprising a housing comprising a first lumen with a proximal opening and a distal opening; a hypodermic needle having a second lumen attached to the housing so that the first and second lumens are fluidically connected via the distal opening; a blood sensor operatively coupled to the first or second lumen to measure the presence of blood in the first or second lumen; and circuitry configured to process data collected by the blood sensor and provide an indicator thereof;
b) inserting the hypodermic needle into a site of the medical procedure;
c) aspirating fluid from the site via the hypodermic needle; and
d) measuring for the presence of blood in the aspirated fluid via the blood sensor to produce data; and
wherein the circuitry analyzes the data and generates an indicator signal representative of the presence or amount of blood.

58. The method of claim 57, wherein the medical procedure is a peripheral nerve block.

59. An apparatus comprising:
a) a constrained flexible cover for a structure; and
b) an application assistance structure connected to the constrained flexible cover,
wherein removal of the application assistance structure allows the constrained flexible cover to assume substantially the same shape as the structure.

60. The apparatus of claim 59, wherein the constrained flexible cover is sterile.

61. The apparatus of claim 59, wherein the application assistance structure is sterile.

62. The apparatus of claim 59, wherein the constrained flexible cover comprises a polymer.

63. The apparatus of claim 59, wherein the polymer is selected from the group consisting of silicone, polyurethane, polytetrafluoroethylene, expanded PTFE, fluorinated ethylene propylene, perfluoroalkoxy, ethylene tetrafluoroethylene, polyvinylidene fluoride, polyethylene, and polypropylene.

64. The apparatus of claim 63, wherein the polymer is silicone.

65. The apparatus of claim 59, wherein the application assistance structure comprises an opening sized to permit the structure to pass.

66. The apparatus of claim 59, wherein the application assistance structure is releasably connected to the constrained flexible cover.

67. The apparatus of claim 59, wherein the structure is a medical device.

68. The apparatus of claim 67, wherein the medical device is the device of any one of claims 1-34.

69. A method of covering a structure, comprising:
a) providing an apparatus comprising:
i) a constrained flexible cover for the structure; and
ii) an application assistance structure connected or connectable to the constrained flexible cover,
b) providing the structure;
c) inserting one end of the structure into the application assistance structure; and
d) moving the application assistance structure to deploy the constrained flexible cover to assume substantially the same shape as the structure.

70. The method of claim 69, wherein the constrained flexible cover is sterile.

71. The method of claim 69, wherein the application assistance structure is sterile.

72. The method of claim 69, wherein the constrained flexible cover comprises a polymer.

73. The method of claim 72, wherein the polymer is selected from the group consisting of silicone, polyurethane, polytetrafluoroethylene, expanded PTFE, fluorinated ethylene propylene, perfluoroalkoxy, ethylene tetrafluoroethylene, polyvinylidene fluoride, polypropylene, and polyethylene.

74. The method of claim 73 wherein the polymer is silicone.

75. The method of claim 69, wherein the application assistance structure comprises an opening sized to permit the structure to pass.

76. The method of claim 69, wherein the application assistance structure is releasably connected to the constrained flexible cover.

77. The method of claim 69, wherein the structure is a medical device.

78. The method of claim 69, wherein the medical device is the device of any one of claims 1-34.

79. A kit, comprising,
a) a structure;
b) a constrained flexible cover for the structure; and
c) an application assistance structure releasably connected or connectable to the constrained flexible cover,
wherein removal of the application assistance structure allows the constrained flexible cover to assume substantially the same shape as the structure.

80. The kit of claim 79, wherein the structure is a medical device.

81. The kit of claim 80, wherein the medical device is the device of any one of claims 1-34.

82. The kit of claim 79, wherein the constrained flexible cover is sterile.

83. The kit of claim 79, wherein the application assistance structure is sterile.

84. The kit of claim 79, wherein the constrained flexible cover comprises a polymer.

85. The kit of claim 84, wherein the polymer is selected from the group consisting of silicone, polyurethane, polytetrafluoroethylene, expanded PTFE, fluorinated ethylene propylene, perfluoroalkoxy, ethylene tetrafluoroethylene, polyvinylidene fluoride, polypropylene, and polyethylene.

86. The kit of claim 85, wherein the polymer is silicone.

87. The kit of claim 79, wherein the application assistance structure comprises an opening sized to permit the structure to pass.
